(19) United States
(12) Patent Application Publication
(10) Pub. No.: US 2005/0282242 A1
(43) Pub. Date: Dec. 22, 2005

(54) Screening assays for antimicrobial agents

(75) Inventors: David M. Rothstein, Lexington, MA (US); Christopher K. Murphy, Upton, MA (US); Ian MacNeil, Milton, MA (US)

Correspondence Address: CLARK & ELBING LLP, 101 FEDERAL STREET, BOSTON, MA, 02110, US

(73) Assignee: ActivBiotics, Inc., Lexington, MA (US)

(21) Appl. No.: 11/115,639

(22) Filed: Apr. 27, 2005

Related U.S. Application Data
(60) Provisional application No. 60/566,858, filed on Apr. 30, 2004.
(60) Provisional application No. 60/565,679, filed on Apr. 27, 2004.

(51) Int. Cl. C12Q 1/18

(52) U.S. Cl. 435/32

(57) ABSTRACT

The present invention provides methods for screening antimicrobial agents that make use of mutated microbial polypeptides. Because these polypeptides have biological activity and result in increased antimicrobial drug resistance, candidate compounds that specifically target these polypeptides provide therapeutics or therapeutic lead compounds for treating infections caused by drug resistant microbial pathogens.

### CROSS REFERENCE TO RELATED APPLICATIONS

This application claims benefit from U.S. Provisional Application Nos. 60/565,679 (filed Apr. 27, 2004) and 60/566,858 (filed Apr. 30, 2004), each of which is hereby incorporated by reference.

### FIELD OF THE INVENTION

The field of the invention relates to drug discovery.

### BACKGROUND OF THE INVENTION

In spite of advances in molecular biology and microbiology, a major difficulty in eradicating infections caused by microbial pathogens is the propensity of such pathogens to rapidly adapt to new environmental challenges and escape the harmful effects of drug therapy. The predominant mechanism of drug resistance is typically caused by mutations in the gene that encodes the protein targeted by the antimicrobial agent. Because these drug resistance-conferring mutations are endogenous (i.e., they require no transfer of DNA from another species), the potential for resistance exists in any sub-population within an infectious population in which the bacterial population number exceeds the mutation frequency.

Rifamycins are a family of chemicals that exhibit potent inhibitory activities against Gram-positive bacteria. Despite their efficacy, the administration of such antibacterial agents may still result in the development of drug resistance, most likely as a result of a mutation in the gene encoding the β subunit of RNA polymerase (RpoB), which contains the rifampin-binding site as defined by X-ray crystal structure (Campbell et al., Cell 104:901-912, 2001).

Due to the constant emergence of drug-resistant microbial strains for the rifamycins and other antibiotics, new antimicrobial agents that are effective against such drug-resistant strains are desirable.

### SUMMARY OF THE INVENTION

In general, the present invention features methods of identifying compounds that inhibit the growth of drug-resistant microbial pathogens. This invention is based on our discovery that antibiotics that specifically target drug resistant bacterial species can be identified using screening methods that employ drug resistance-conferring polypeptides. We show, for example, that rifampin derivatives that specifically target rifampin-resistant bacteria can be identified using screening assays that identify compounds that target the mutated β subunit of RNA polymerase. Accordingly, antimicrobial agents that inhibit the growth of drug-resistant pathogens are identified on the basis of their ability to bind and/or decrease the biological activity or expression level of drug resistance-conferring microbial polypeptides. Our results further show that screening methods that make use of a plurality of drug resistance-conferring polypeptides allow for the identification of antimicrobial agents associated with an improved ability to specifically and effectively inhibit the growth of drug-resistant microbial pathogens.

According to this invention, a compound that inhibits the growth of drug resistant microbial pathogens may be identified by a method involving the steps of: (a) producing a derivative compound of an antimicrobial compound; (b) contacting the derivative compound with a plurality of mutated microbial polypeptides conferring drug resistance, under conditions that ensure that each contacting event is segregated from the others; and (c) determining whether the derivative compound interacts with the mutated microbial polypeptides. A derivative compound that interacts with at least two different mutated microbial polypeptides is identified as a compound that inhibits the growth of drug resistant microbial pathogens. In one example, a compound having weak antimicrobial activity may be used as a lead compound for the design of improved antimicrobial agents. Derivative compounds are produced using information provided by the lead compound and these derivative compounds are screened for their antimicrobial activity. Using the methods of the invention, compounds having increased antimicrobial activity (e.g., at least 10%, 20%, 30%, 40%, 50%, 60%, 70%, 80%, 90%, or 100%) relative to the lead compound and having the ability to reduce the growth of drug resistant microbial pathogens may be identified.

The invention also features a method of identifying a compound that inhibits the growth of drug resistant microbial pathogens, involving the steps of: (a) contacting at least 10, 20, 30, 40, 50, 60, 80, 100, or more than 100 candidate compounds with a plurality of mutated microbial polypeptides conferring drug resistance, under conditions that ensure that each contacting event is segregated from the others; and (b) determining whether the candidate compounds interact with the mutated microbial polypeptides. A candidate compound that interacts with at least two different mutated microbial polypeptides is identified as a compound that inhibits the growth of drug resistant microbial pathogens.

Alternatively, the invention features a method of identifying a compound that inhibits the growth of drug resistant microbial pathogens, involving the steps of: (a) contacting a candidate compound with a plurality of mutated microbial polypeptides conferring drug resistance under conditions that ensure that each contacting event is segregated from the others; and (b) determining whether the candidate compound binds the mutated microbial polypeptides. A candidate compound that binds at least two different mutated microbial polypeptides is identified as a compound that inhibits the growth of drug resistant microbial pathogens.

Alternatively, the invention features a method of identifying a compound that inhibits the growth of drug resistant microbial pathogens, involving the steps of: (a) contacting a candidate compound with a plurality of mutated microbial polypeptides conferring drug resistance under conditions that ensure that each contacting event is segregated from the others; and (b) determining in vitro whether the candidate compound reduces the biological activity of the mutated microbial polypeptides. A candidate compound that reduces the biological activity of at least two different mutated microbial polypeptides is identified as a compound that inhibits the growth of drug resistant microbial pathogens.

A compound that inhibits the growth of drug resistant microbial pathogens may also be identified using a method involving the steps of: (a) contacting a candidate compound with a mutated microbial polypeptide conferring drug resistance in vitro; (b) determining whether the candidate compound interacts with the mutated microbial polypeptide, and continuing to step (c) if the candidate compound interacts with the mutated microbial polypeptides; (c) contacting the candidate compound with a mutated microbial polypeptide in an animal; and (d) determining whether the candidate compound interacts with the mutated microbial polypeptide in the animal. A candidate compound that interacts with the mutated microbial polypeptide in the animal is identified as a compound that inhibits the growth of drug resistant microbial pathogens.

The invention also features a method of identifying a compound that inhibits the growth of drug resistant microbial pathogens, involving the steps of: (a) contacting a candidate compound with a mutated microbial polypeptide conferring drug resistance; (b) determining whether the candidate compound interacts with the mutated microbial polypeptide, and continuing to step (c) if the candidate compound interacts with the mutated microbial polypeptide; (c) contacting the candidate compound with a plurality of mutated microbial polypeptides conferring drug resistance under conditions that ensure that each contacting event is segregated from the others; and (d) determining whether the candidate compound interacts with the mutated microbial polypeptides, such that a candidate compound that interacts with at least two different mutated microbial polypeptides is identified as a compound that inhibits the growth of drug resistant microbial pathogens.

For each of the above methods, a candidate compound is identified as a compound that inhibits the growth of drug resistant microbial pathogens if it interacts, binds, or reduces the biological activity of at least two, three, four, five, six, ten, twenty, or more than twenty mutated microbial polypeptides. If desired, the mutated microbial polypeptide may be operably linked to a reporter gene in any of the methods of the invention. A candidate compound may therefore be identified as a useful compound to inhibit the growth of drug resistant pathogens based on its ability to reduce expression of the reporter gene. Furthermore, the contacting event between a candidate compound and a mutated microbial polypeptide may occur inside a cell (e.g., microbial cell) or in a cell-free environment. The contacting event may therefore occur in an intracellular pathogen such as an obligate intracellular pathogen or a facultative intracellular pathogen. If an intracellular pathogen is employed in the present methods, the host of the pathogen may also be present. Obligate intracellular pathogens include bacteria, protozoans, and fungi. Obligate intracellular bacteria include, for example, Anaplasma bovis, A. caudatum, A. centrale, A. marginale A. ovis, A. phagocytophila, A. platys, Bartonella bacilliformis, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, and R. typhi. Exemplary intracellular protozoans are Brachiola vesicularum, B. connori, Encephalitozoon cuniculi, E. hellem, E. intestinalis, Enterocytozoon bieneusi, Leishmania aethiopica, L. amazonensis, L. braziliensis, L. chagasi, L. donovani, L. donovani chagasi, L. donovani donovani, L. donovani infantum, L. enriettii, L. guyanensis, L. infantum, L. major, L. mexicana, L. panamensis, L. peruviana, L. pifanoi, L. tarentolae, L. tropica, Microsporidium ceylonensis, M. africanum, Nosema connori, Nosema ocularum, N. algerae, Plasmodium berghei, P. brasilianum, P. chabaudi, P. chabaudi adami, P. chabaudi chabaudi, P. cynomolgi, P. falciparum, P. fragile, P. gallinaceum, P. knowlesi, P. lophurae, P. malariae, P. ovale, P. reichenowi, P. simiovale, P. simium, P. vinckeipetteri, P. vinckei vinckei, P. vivax, P. yoelii, P. yoelii nigeriensis, P. yoelii yoelii, Pleistophora anguillarum, P. hippoglossoideos, P. mirandellae, P. ovariae, P. typicalis, Septata intestinalis, Toxoplasma gondii, Trachipleistophora hominis, T. anthropophthera, Vittaforma corneae, Trypanosoma avium, T. brucei, T. brucei brucei, T. brucei gambiense, T. brucei rhodesiense, T. cobitis, T. congolense, T. cruzi, T. cyclops, T. equiperdum, T. evansi, T. dionisii, T godfreyi, T. grayi, T. lewisi, T. mega, T. microti, T. pestanai, T. rangeli, T. rotatorium, T. simiae, T. theileri, T. varani, T. vespertilionis, and T. vivax. Furthermore, exemplary obligate intracellular fungi are Histoplasma capsulatum or a species of the genus Candida. If desired, the contacting event may occur in vivo. Accordingly, an animal having an infection with microbial pathogens that express mutated polypeptides conferring drug resistance may be treated with a candidate compound.

Interactions between the candidate compound and the mutated microbial polypeptide conferring drug resistance may be determined by any standard method known in the art including, for example, the determination of microbial cell growth, biological activity of the mutated microbial polypeptide, or binding between the candidate compound and the mutated microbial polypeptide. If the contacting event occurs in vivo (i.e. by application of the candidate compound on or in the animal by any route of administration (e.g., topical, oral, dermal, sub-cutaneous, intraperitoneal, and intravenous administration)), interaction between the candidate compound and the mutated microbial polypeptide may be determined using any standard method known in the art, including for example, survival assays or assays that detect microbial load (e.g., bacterial load in a biological sample from the animal). Thus, a useful candidate compound reduces the number of microbial pathogens in said animal (by at least 10%, 20%, 30%, 40%, 50%, 60%, 70%, 80%, 90% or 100% relative to an untreated control), increases the survival of the animal (by at least 10%, 20%, 30%, 40%, 50%, 60%, 70%, 80%, 90% or 100% relative to an untreated control), or both. Exemplary methods for each of these methods are provided herein. Others are well known in the art.

Mutations in microbial polypeptides may occur, for example, at any site where an antimicrobial agent typically binds. A microbial cell expressing such a mutated microbial polypeptide in lieu of its wild-type counterpart is resistant to an antimicrobial agent. In the case of RpoB polypeptides, for example, drug resistance-conferring mutations often occur in the rifampin-binding site within the β subunit. Mutated RpoB polypeptides may have a mutation at one or more of the amino acid positions corresponding to amino acid positions 137, 464, 466, 468, 471, 477, 481, 484, 486, and 527 of S. aureus RpoB. Exemplary S. aureus mutations are Q137L, S464P, L466S, Q468R, Q468K, D471V, D471Y, D471G, D471E, A477V, A477D, H481D, H481R, H481Y, H481N, R484H, R484S, R484C, S486L, I527P, and I527M. Mutated microbial polypeptides may be derived from any microbial pathogen (e.g., bacterium (e.g., a Gram-positive bacterium), fungus, virus, or parasite). Exemplary bacteria are Bacillus anthracis, Bacillus cereus, Bacillus subtilis, Chlamydia pneumoniae, Chlamydia trachomatis, Enterococcus faecium, Escherichia coli, Haemophilus influenzae, Helicobacter pylori, Listeria monocytogenes, Mycobacterium tuberculosis, Neisseria meningitidis, Staphylococcus aureus, Streptococcus pneumoniae, and Streptococcus pyogenes.

The invention also features a surface on which a plurality of mutated microbial polypeptides conferring drug resistance is arrayed. The polypeptides may be within a bacterial cell or may be in a cell-free environment. The mutated microbial polypeptides are arranged on the surface such that, when contacted with a candidate compound, each polypeptide-candidate compound contacting event is segregated from the others. The polypeptides may be in solution (e.g., each in its own well of a multiwell plate) or may be immobilized on the surface (e.g., in wells of a multiwell plate or on a slide). Desirably, the polypeptides are mutated RpoB polypeptides. In a related aspect, the invention also features a plurality of chromatographic columns, wherein each column has a mutated microbial polypeptide conferring drug resistance (e.g., a mutated RpoB polypeptide).

The invention further features a method of measuring RNA polymerase activity that makes use of molecular beacon probes. Such a probe is an oligonucleotide molecule that is covalently linked to a quencher at the 5′ or 3′end and to a fluorophore at the opposite end. The probe contains a nucleotide sequence that forms a hairpin structure having a stem region that contains a double stranded segment formed between two complementary nucleotide sequences under suitable conditions. The formation of such a double stranded segment brings the fluorophore and quencher into close proximity, resulting in inhibition or reduction in fluorescence emission by the fluorophore. The method of the invention involves the steps of: (a) providing the molecular beacon probe of the invention described above; (b) contacting this probe with a test sample under conditions allowing transcription from the probe; and (c) measuring the level of fluorescence emission from the test sample relative to a control sample, such that an increase in fluorescence identifies the test sample as containing RNA polymerase polypeptides associated with biological activity. According to our assay, in the presence of a biologically active RNA polymerase polypeptide, an RNA transcript is produced from the probe. This RNA transcript binds to the complementary probe producing a RNA:DNA hybrid that disrupts the double stranded stem region of the probe. This disruption causes the fluorophore and quencher to physically separate, resulting in an increase in the emission of fluorescence from the fluorophore. An essential feature of the assay is that the transcription template is the molecular beacon probe rather than any other templates that may be present in the sample. This assay is therefore useful to detect RNA polymerase activity in any of the mutated RNA polymerase polypeptides of the invention (e.g., RpoB polypeptides). Desirably, the increase in the emission of fluorescence is at least 10%, 20%, 30%, 40%, 50%, 60%, 70%, 80%, 90%, 100%, or more than that of a control sample. The measurement of fluorescence is standard in the art and is described, for example, by Liu et al. (Anal. Biochem. 300:40-45, 2002).

The above method is also useful, for example, to identify a candidate compound as having the ability to inhibit the growth of drug resistant microbial pathogens. In this method, a candidate compound is contacted with one or more than one mutated microbial polypeptides (e.g., RNA polymerase, preferably containing an RpoB subunit) conferring drug resistance and the molecular beacon probe described above. If a plurality of mutated microbial polypeptides is employed in the present screening methods, the contacting event occurs under conditions that ensure that each contacting event is segregated from the others. The biological activity of RNA polymerase is determined in each contacting event using the method described above. A candidate compound that reduces the biological activity of at least two mutated microbial polypeptides is identified as a compound having the ability to reduce the growth of drug resistant microbial pathogens.

A microbial pathogen expressing a mutant microbial polypeptide is considered “drug resistant” if it has an increased ability to withstand the harmful or toxic effects of at least one antimicrobial agent relative to its wild-type counterpart, as measured by any standard method in the art. Accordingly, the growth rate of the drug resistant pathogen in the presence of an antimicrobial agent may be at least 10%, 20%, 30%, 40%, 50%, 60%, 70%, 80%, 90%, or 100% greater than that of the wild-type microbial pathogen. Alternatively, a drug resistant microbial pathogen includes those for which the ability of the antimicrobial agent to inhibit infection or growth is reduced by at least 10%, 20%, 30%, 40%, 50%, 60%, 70%, 80%, 90%, or 100% relative to the wild-type pathogen, as measured by any standard method such as those described herein (e.g., MIC assay).

Compounds “having antimicrobial properties against drug resistant microbial pathogens” are those that inhibit infection or the growth of such pathogens. Such inhibition may be at least 10%, 20%, 30%, 40%, 50%, 60%, 70%, 80%, 90%, or 100%, relative to an untreated control.

By a “mutated microbial polypeptide conferring drug resistance” is meant that the polypeptide contains at least one mutation (e.g., an amino acid substitution, insertion, or deletion) but nonetheless exhibits an activity common to its related, wild-type microbial polypeptide. The activity may be at levels that are reduced relative to the wild-type polypeptide. When the mutated polypeptide is expressed in a microbial organism in lieu of its wild-type counterpart, the microbial organism exhibits drug resistance. Accordingly, the ability of the organism to withstand the toxic effects of at least one antimicrobial agent is increased by at least 10%, 20%, 30%, 40%, 50%, 60%, 70%, 80%, 90%, or 100%, relative to that of the wild-type. A mutated microbial polypeptide is considered to “have biological activity” if it has at least 20%, 30%, 40%, 50%, 60%, 70%, 80%, 90%, or 100% of the biological activity of the naturally occurring microbial polypeptide as measured by any standard method. For example, one of the biological activities of the naturally occurring RNA polymerase polypeptide is the production of RNA from a DNA template.

By “RNA polymerase polypeptide” is meant a polypeptide that is substantially identical to a portion of or the entire sequence of a polypeptide subunit of a naturally occurring RNA polymerase. Accordingly, the RNA polymerase polypeptide of the invention need not be substantially identical to the full length, naturally occurring RNA polymerase but may simply be substantially identical to a portion within the full length sequence. Desirably, the RNA polymerase polypeptide contains a sequence that is substantially identical to the β subunit of the wild type RNA polymerase.

By “substantially identical,” when referring to a protein or polypeptide, is meant a protein or polypeptide exhibiting at least 75%, but preferably 85%, more preferably 90%, most preferably 95%, or even 99% identity to a reference amino acid sequence. For proteins or polypeptides, the length of comparison sequences will generally be at least 10, 20, 30, 40, 50, 60, 70, 80, 90, 100, 150, 200, 300 amino acids, or the full length protein or polypeptide. Nucleic acids that encode such “substantially identical” proteins or polypeptides constitute an example of “substantially identical” nucleic acids; it is recognized that the nucleic acids include any sequence, due to the degeneracy of the genetic code, that encodes those proteins or polypeptides. In addition, a “substantially identical” nucleic acid sequence also includes a polynucleotide that hybridizes to a reference nucleic acid molecule under high stringency conditions.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference.

By a “candidate compound” is meant a chemical, be it naturally-occurring or artificially-derived. Candidate compounds may include, for example, peptides, polypeptides, peptide nucleic acids, synthetic organic molecules, naturally occurring organic molecules, nucleic acid molecules, and components thereof.

By a “derivative compound of an antimicrobial compound” is meant a chemical (e.g., peptides, polypeptides, peptide nucleic acids, synthetic organic molecules, naturally occurring organic molecules, nucleic acid molecules, and components thereof) that shares chemical, structural, or functional similarities with a compound known to have antimicrobial activity. The antimicrobial compound (from which the derivative compound is produced) may or may not be used as the starting material for the production of the derivative compound. Accordingly, the antimicrobial compound may simply be required to provide chemical, structural, or functional information for the production of the derivative compound, thereby functioning as a lead compound in the design of improved antimicrobial compounds.

By “amino acid position corresponding to S. aureus RpoB position X” is meant that an amino acid is located in an RpoB polypeptide at a position analogous to position X of S. aureus RpoB. For various bacteria, amino acid positions corresponding to S. aureus RpoB position X are shown in Table 1 and FIGS. 1A-1B. Other analogous positions may be determined by aligning the desired RpoB polypeptide with S. aureus RpoB (GenBank Accession No. 15926220) using BLAST2 (Tatiana et al., FEMS Microbiol. Lett. 174:247-250, 1999) and default parameters (Matrix: BLOSUM62 (Henikoff et al., Proc. Natl. Acad. Sci. 89: 10915-10919, 1992); gap open: 11; gap extension: 1; x_dropoff: 30; expect: 10; wordsize: 3; filter: yes).

By “polypeptide” is meant any chain of more than two amino acids, regardless of post-translational modification such as glycosylation or phosphorylation.

By “substantially pure polypeptide” is meant a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is at least 75%, more preferably at least 90%, and most preferably at least 99%, by weight, pure. A substantially pure polypeptide may be obtained by any standard method (as described herein), for example, by extraction from a natural source, by expression of a recombinant nucleic acid encoding the polypeptide, or by chemical synthesis of the polypeptide. Purity may be measured by any appropriate method, e.g., by column chromatography, polyacrylamide gel electrophoresis, or HPLC analysis.

A polypeptide is substantially free of naturally associated components when it is separated from those contaminants that accompany it in its natural state. Thus, a polypeptide that is chemically synthesized or produced in a cellular system different from the cell from which it naturally originates will be substantially free from its naturally associated components. Accordingly, substantially pure polypeptides include those that naturally occur in eukaryotic organisms but are synthesized in E. coli, yeast, or other microbial system.

By “obligate intracellular pathogen” is meant a microbe that must use an intracellular location (e.g., a host cell) in order to replicate.

By “facultative intracellular pathogen” is meant a microbe that is able to survive within an intracellular location (e.g., a host cell), but does not require an intracellular environment to replicate.

TABLE 1
Amino acid positions in bacteria corresponding to S. aureus RpoB positions

| | Organism GenBank Accession No. | | | | | | | | |
|---|---|---|---|---|---|---|---|---|---|
| | S. aureus | B. anthracis | B. cereus | B. subtilis | C. pneumoniae | C. trachomatis | C. perfringens | E. Coli | E. faecalis |
| | 15926220 | 30260293 | 42779183 | 585920 | 8978454 | 6831647 | 18146079 | 13364386 | 41017700 |
| Amino | 135 | 135 | 135 | 135 | 136 | 136 | 135 | 146 | 138 |
| acid | 137 | 137 | 137 | 137 | 138 | 138 | 137 | 148 | 140 |
| position | 464 | 464 | 464 | 465 | 454 | 454 | 485 | 509 | 472 |
| | 466 | 466 | 466 | 467 | 456 | 456 | 487 | 511 | 474 |
| | 467 | 467 | 467 | 468 | 457 | 457 | 488 | 512 | 475 |
| | 468 | 468 | 468 | 469 | 458 | 458 | 489 | 513 | 476 |
| | 471 | 471 | 471 | 472 | 461 | 461 | 492 | 516 | 479 |
| | 477 | 477 | 477 | 478 | 467 | 467 | 498 | 522 | 485 |
| | 481 | 481 | 481 | 482 | 471 | 471 | 502 | 526 | 489 |
| | 484 | 484 | 484 | 485 | 474 | 474 | 505 | 529 | 492 |
| | 486 | 486 | 486 | 487 | 476 | 476 | 507 | 531 | 494 |
| | 527 | 527 | 527 | 528 | 517 | 517 | 548 | 572 | 535 |
| | 571 | 571 | 571 | 572 | 559 | 559 | 592 | 614 | 579 |
| | 651 | 651 | 651 | 652 | 639 | 639 | 672 | 694 | 659 |
| | 665 | 665 | 665 | 666 | 653 | 653 | 686 | 708 | 673 |

Organism GenBank Accession No.

| | E. faecium | H. influenzae | H. pylori | L. monocytogenes | M. tuberculosis | N. meningitidis | S. pneumoniae | S. pyogenes |
|---|---|---|---|---|---|---|---|---|
| | 41017745 | 1173148 | 15645812 | 6002201 | 13880218 | 15676060 | 15903819 | 21903792 |
| Amino | 138 | 146 | 149 | 138 | 176 | 152 | 148 | 135 |
| acid | 140 | 148 | 151 | 140 | 178 | 154 | 150 | 137 |
| position | 472 | 509 | 523 | 466 | 434 | 539 | 482 | 469 |
| | 474 | 511 | 525 | 468 | 436 | 540 | 484 | 471 |
| | 475 | 512 | 526 | 469 | 437 | 541 | 485 | 472 |
| | 476 | 513 | 527 | 470 | 438 | 542 | 486 | 473 |
| | 479 | 516 | 530 | 473 | 441 | 545 | 489 | 476 |
| | 485 | 522 | 536 | 479 | 447 | 551 | 495 | 482 |
| | 489 | 526 | 540 | 483 | 451 | 555 | 499 | 486 |
| | 492 | 529 | 543 | 486 | 454 | 558 | 502 | 489 |
| | 494 | 531 | 545 | 488 | 456 | 560 | 504 | 491 |
| | 535 | 572 | 586 | 529 | 497 | 601 | 545 | 532 |
| | 579 | 614 | 628 | 573 | 539 | 643 | 589 | 576 |
| | 659 | 695 | 708 | 653 | 620 | 723 | 669 | 656 |
| | 673 | 709 | 722 | 667 | 634 | 737 | 683 | 670 |

By “fusion protein” is meant a first polypeptide fused to a second, heterologous polypeptide. For example, the mutated microbial polypeptide of the invention may be fused to a second, heterologous polypeptide.

By “reporter polypeptide” is meant one whose expression may be specifically assayed. Reporter polypeptides include, without limitation, glucuronidase (GUS), luciferase, chloramphenicol transacetylase (CAT), green fluorescent protein (GFP), alkaline phosphatase, and β-galactosidase.

By “specifically binds” is meant that a small molecule, peptide, antibody, or polypeptide binds a second small molecule, peptide, antibody, or polypeptide but does not substantially recognize and bind other molecules in a sample, e.g., a biological sample. Desirably, such binding is at least 10%, 20%, 30%, 40%, 50%, 60%, 70%, 80%, 90%, or 100% greater than binding to non-specific sample components.

The predominant mechanism of microbial drug resistance is largely attributed to mutations in the genes encoding microbial polypeptides that are targeted by antimicrobial drugs. The present invention features screening methods for identifying antimicrobial agents that inhibit the growth of drug resistant microbial agents. Because these screening assays specifically identify compounds that bind and/or reduce the expression level or biological activity of drug resistance-conferring polypeptides, antimicrobial agents having the ability to target drug resistant microbial pathogens can be readily detected. In particular, the invention provides screening methods that make use of a plurality of drug resistance-conferring polypeptides. The use of a panel of such polypeptides results in the fine-tuning of antimicrobial agents that can specifically and effectively inhibit the growth of drug resistant microbial pathogens.

Other features and advantages of the invention will be apparent from the following detailed description and from the claims.

### BRIEF DESCRIPTION OF THE DRAWINGS

FIGS. 1A and 1B show a schematic diagram indicating RpoB mutations in various microbial pathogens.

FIG. 2 is a schematic diagram of an assay that utilizes molecular beacons as probes to measure transcription.

FIG. 3 is a schematic diagram of a second assay that measures transcription.

FIG. 4 is a graph showing the activity of RNA polymerase in the presence of rifampicin and rifalazil.

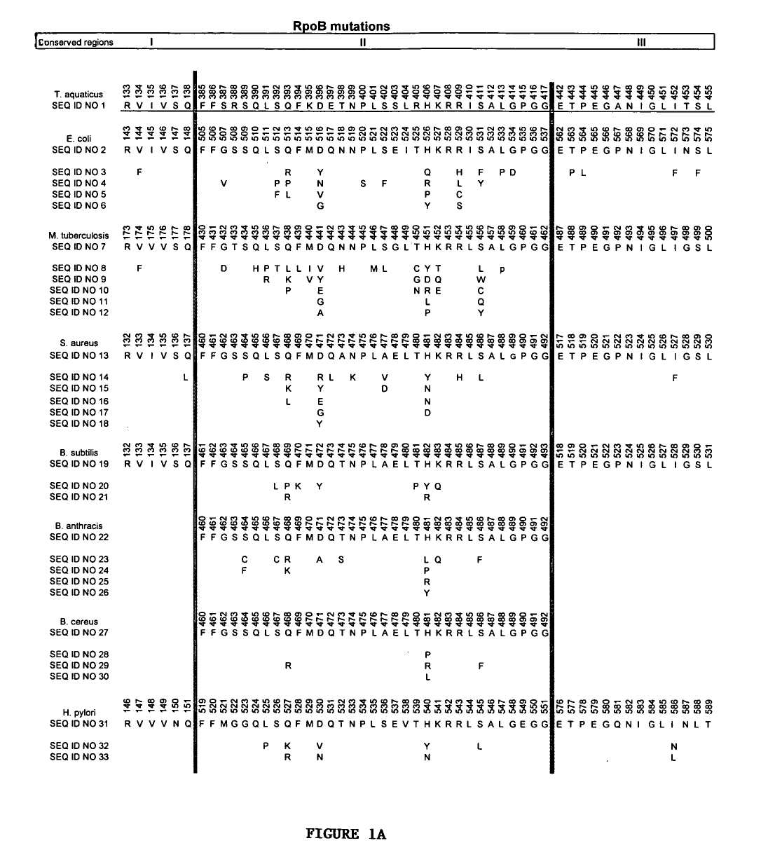

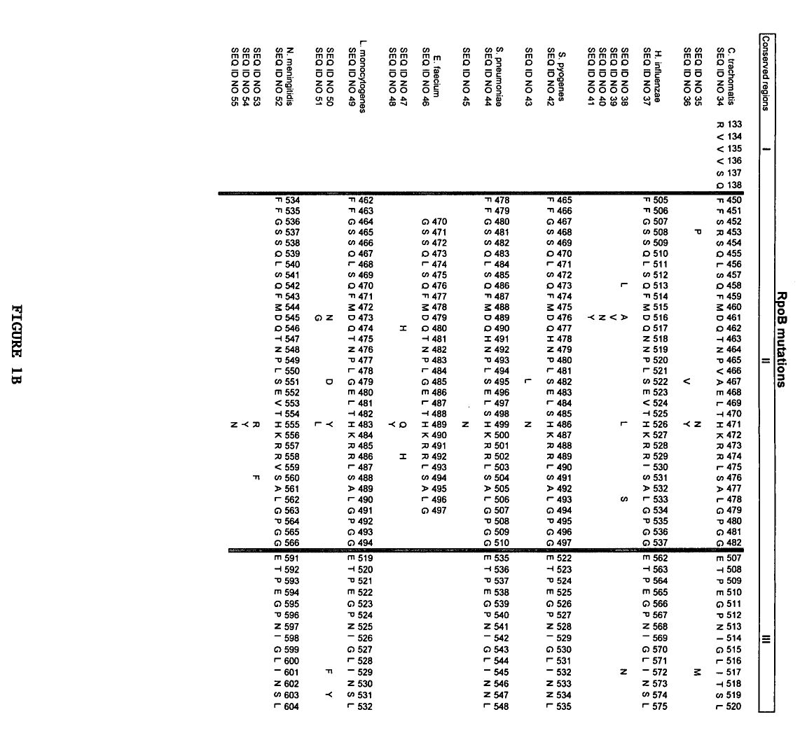

### DETAILED DESCRIPTION

The increasing rate of microbial drug resistance is largely attributed to the ability of pathogens to rapidly adapt to environmental pressures. Upon drug exposure, drug resistant mutants emerge through the natural selection of microbial species in which the microbial polypeptides targeted by antimicrobial drugs have been mutated. In spite of their antibacterial efficacy, rifamycins, for example, can eventually become less effective as drug resistant mutants emerge. Resistance to rifamycins typically occurs as a result of a mutation in the gene encoding the β subunit of RNA polymerase (RpoB), which contains the rifampin-binding site as defined by X-ray crystal structure.

Here, we describe screening methods that make use of a comprehensive panel of bacterial mutant strains resistant to rifamycins to identify compounds having improved interactions with mutated target bacterial RNA polymerase polypeptides. Using these methods, antibacterial agents that function as inhibitors of the mutated RNA polymerases are identified. Because antimicrobial agents are identified based specifically on their ability to interact with the microbial polypeptides conferring drug resistance, antimicrobial agents that overcome that resistance may be isolated. Using this general approach, a wide range of new antimicrobials may be identified by using mutated polypeptides that confer resistance to other antibiotics.

Screening Assays

As indicated above, in the present assays, candidate compounds are screened for their ability to interact with mutated microbial polypeptides. Particularly useful candidate compounds have the ability to interact with a plurality of mutated microbial polypeptides, thereby reducing or inhibiting the biological activity of such polypeptides. Thus, these methods desirably employ a plurality of mutated microbial polypeptides (e.g., at least 2, 3, 4, 5, 10, 15, 20, 30, 40, or more than 40 different mutated microbial polypeptides). These polypeptides may be different mutants of the same wild-type microbial polypeptide, or alternatively, mutants of different wild-type microbial polypeptides. Furthermore, a candidate compound may be contacted with various different, mutated RpoB polypeptides derived from two or more different bacterial species. A candidate compound is identified as being an antimicrobial compound if it interacts with at least one, two, or more mutated microbial polypeptides. The present methods are useful for screening compounds having an effect on a variety of microbial organisms, including, but not limited to, bacteria, viruses, fungi, annelids, nematodes, platyhelminthes, and protozoans.

Interactions between candidate compounds and mutated microbial polypeptides may be assessed by any standard method, such as those that measure or detect direct binding, competitive binding, enzymatic activity, cell growth, or transcription. The screen may initially involve a pool of candidate compounds, from which one or more useful compounds are isolated in a step-wise fashion. Desirably, the testing of unknown compounds involves high throughput screens (see, for example, Williams, Medicinal Research Reviews, 11:147-184, 1991; Sweetnam, et al., J. Natural Products, 56:441-455, 1993).

Overall, the invention provides a simple means for identifying antimicrobial compounds (including peptides, small molecule inhibitors, and mimetics) effective against drug resistant microbial pathogens. Accordingly, a chemical entity discovered to have medicinal or agricultural value using the methods described herein are useful as either drugs, plant protectants, or as information for structural modification of existing anti-pathogenic compounds, e.g., by rational drug design. Compounds isolated by this approach may be used, for example, as therapeutics to treat or prevent a microbial infection.

Microbial Polypeptide Expression and Purification

For their use in the present invention, recombinant mutated microbial polypeptides may be produced using any standard technique known in the art. Following their production, these polypeptides are useful, for example, for the identification of therapeutic compounds using the methods described herein.

Host cells, such as yeast, bacterial, mammalian, and insect cells, may produce any of the polynucleotides of the present invention. These cells may produce such polynucleotides endogenously or may alternatively be genetically engineered to do so. Polynucleotides may be introduced into host cells using any standard method known in the art, including, for example, calcium phosphate transfection, DEAE-dextran mediated transfection, transvection, microinjection, cationic lipid-mediated transfection, electroporation, transduction, ballistic introduction, and infection or fusion with carriers such as liposomes, micelles, ghost cells, and protoplasts.

In general, any expression system or vector that is able to maintain, propagate, or express a polynucleotide to produce a polypeptide in a host may be used. These include chromosomal, episomal, and virus-derived systems such as vector-derived bacterial plasmids, bacteriophages, transposons, yeast episomes, insertion elements, yeast chromosomal elements, viruses (such as baculoviruses, papova viruses (e.g., SV40), vaccinia viruses, adenoviruses, fowl pox viruses, pseudorabies viruses, and retroviruses), and vectors derived from combinations thereof, such as those derived from plasmid and bacteriophage genetic elements, such as cosmids and phagemids. Preferred expression vectors include, but are not limited to, pcDNA3 (Invitrogen) and pSVL (Pharmacia Biotech). Other exemplary expression vectors include pSPORT vectors, pGEM vectors (Promega), pPROEXvectors (LTI, Bethesda, Md.), Bluescript vectors (Stratagene), pQE vectors (Qiagen), pSE420 (Invitrogen), and pYES2 (Invitrogen). Optionally, the expression systems may contain control regions that facilitate or regulate expression. The appropriate polynucleotide may be inserted into an expression system by any of a variety of well-known and routine techniques, including transformation, transfection, electroporation, nuclear injection, or fusion with carriers such as liposomes, micelles, ghost cells, and protoplasts.

Expression systems of the invention include bacterial, yeast, fungal, plant, insect, invertebrate, vertebrate, and mammalian cells systems. If a eukaryotic expression vector is employed, then the appropriate host cell is any eukaryotic cell capable of expressing the cloned sequence. Preferably, eukaryotic cells are cells of higher eukaryotes. Suitable eukaryotic cells include non-human mammalian tissue culture cells and human tissue culture cells. Preferred host cells include insect cells, HeLa cells, Chinese hamster ovary cells (CHO cells), African green monkey kidney cells (COS cells), human 293 cells, murine embryonal stem (ES) cells, and murine 3T3 fibroblasts. The propagation of such cells in cell culture is standard in the art. Yeast hosts may also be employed as a host cell. Preferred yeast cells include the genera Saccharomyces, Pichia, and Kluveromyces. Preferred yeast hosts are Saccharomyces cerevisiae and Pichia pastoris. Yeast vectors may contain any of the following elements: an origin of replication sequence from a 2T yeast plasmid, an autonomous replication sequence (ARS), a promoter region, sequences for polyadenylation, sequences for transcription termination, and a selectable marker gene. Shuttle vectors for replication in both yeast and E. coli are also included herein.

Alternatively, insect cells may be used as host cells. In a preferred embodiment, the polypeptides of the invention are expressed using a baculovirus expression system. The Bac-to-Bac complete baculovirus expression system (Invitrogen) may be used, for example, for protein production in insect cells.

Expression of polypeptides in prokaryotes is most often carried out in E. coli with vectors containing constitutive or inducible promoters directing the expression of either fusion or non-fusion proteins. Fusion vectors add a number of amino acids to a polypeptide encoded therein, usually to the amino terminus of the recombinant polypeptide. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant polypeptide to enable separation of the recombinant protein from the fusion moiety subsequent to purification of the fusion protein. Such enzymes, and their cognate recognition sequences, include Factor Xa, thrombin, and enterokinase. Typical fusion expression vectors include pGEX, pMAL, and pRIT5, which fuse glutathione S-transferase (GST), maltose E binding protein, and protein A, respectively, to the target recombinant protein.

The polypeptides of the present invention may also be expressed at the surface of cells, which are then harvested prior to use in the screening assay. If the polypeptide is secreted into the medium, the medium may be recovered in order to recover and purify the polypeptide. If produced intracellularly, the cells must first be lysed before the polypeptide is recovered. Polypeptides of the present invention may be recovered and purified from recombinant cell cultures or lysates by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography, and lectin chromatography. Most preferably, high performance liquid chromatography is employed for purification. Well-known techniques for refolding proteins may be employed to regenerate active conformation when the polypeptide is denatured during intracellular synthesis, isolation, and/or purification.

Optionally, the polypeptides of the present invention may be prepared by chemical synthesis using, for example, automated peptide synthesizers.

Candidate Compounds

Candidate compounds (e.g., organic molecules, peptides, peptide mimetics, polypeptides, nucleic acids, or antibodies) may be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (see e.g., Lam, Anticancer Drug Des. 12:145, 1997).

Examples of methods for the synthesis of molecular libraries can be found in the art, for example in: DeWitt et al., Proc. Natl. Acad Sci. USA. 90:6909, 1993; Erb et al., Proc. Natl. Acad Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Libraries of compounds may be presented in solution (Houghten, Biotechniques 13:412-421, 1992), or on beads (Lam, Nature 354:82-84, 1991), chips (Fodor, Nature 364:555-556, 1993), bacteria (Ladner U.S. Pat. No. 5,223,409), spores (Ladner U.S. Pat. No. 5,223,409), plasmids (Cull et al., Proc. Natl. Acad. Sci. USA 89:1865-1869, 1992) or on phage (Scott et al., Science 249:386-390, 1990; Devlin, Science 249:404-406, 1990; Cwirla et al., Proc. Natl. Acad. Sci. 87:6378-6382 1990; Felici, J. Mol. Biol. 222:301-310, 1991).

Optionally, either the mutated microbial polypeptide or the candidate compound may include a label or tag that facilitates their isolation. For polypeptides, an exemplary tag of this type is a poly-histidine sequence generally containing around six histidine residues that permits the isolation of a compound so labeled by means of nickel chelation. Other labels and tags, such as the FLAG tag (Eastman Kodak, Rochester, N.Y.), are well known and are routinely used in the art. Small molecules may be radiolabeled for detection.

Interaction Assays

One method to identify antimicrobial agents involves screening for compounds that physically interact with mutated microbial polypeptides. Such compounds are identified as being candidate antimicrobial compounds effective against drug-resistant microbial pathogens. Recombinant mutated microbial polypeptides (produced by any standard methods such as those as described above) are preferred for binding assays, particularly in high-throughput screens because they allow for better specificity (higher relative purity), provide the ability to generate large amounts of material, and can be used in a broad variety of formats (see, e.g., Hodgson, Bio/Technology, 10:973-980, 1992).

Binding may be determined by various assays well known in the art, including gel-shift assays, western blots, radiolabeled competition assay, phage-based expression cloning, co-fractionation by chromatography, co-precipitation, cross-linking, interaction trap/two-hybrid analysis, southwestern analysis, ELISA, and the like, which are described, for example, in Current Protocols in Molecular Biology, 2001, John Wiley & Sons, NY, which is incorporated herein by reference.

As discussed above, in any of the foregoing assays, the mutated microbial polypeptide or the candidate compound may be labeled with a detectable label to facilitate the detection of binding. In some instances, it may be desirable to immobilize the mutated microbial polypeptide(s) or the candidate compound(s). Immobilization may be accomplished using any of the methods well known in the art, including covalent bonding to a support, a bead, or a chromatographic resin; non-covalent, high affinity interactions such as antibody binding; or use of streptavidin/biotin binding such that the immobilized compound includes a biotin moiety. Thus, the detection of binding may be accomplished using (i) a radioactive label on the compound that is not immobilized, (ii) a fluorescent label on the non-immobilized compound, (iii) an antibody immunospecific, for the non-immobilized compound, or (iv) a label on the non-immobilized compound that excites a fluorescent support to which the immobilized compound is attached.

In one embodiment, the screening method of the invention includes the steps of (a) contacting one or more mutated microbial polypeptides with one or more candidate compounds; and (b) measuring binding between the compound(s) and mutated microbial polypeptide(s). Desirably, a plurality of mutated microbial polypeptides is employed, in which case each contacting event is physically separated from the others. Binding may be measured directly (e.g., by using a labeled compound as described above) or indirectly using any of a number of techniques. Following steps (a) and (b), compounds identified as binding a mutated microbial polypeptide may be further tested in other assays, including assays of biological activity or cell growth.

As a specific example, a candidate compound that binds a mutated microbial polypeptide may be identified using a chromatography-based technique. Accordingly, a recombinant mutated microbial polypeptide, such as S. aureus RpoB containing a Q468K mutation, may be purified by standard techniques from cells engineered to express the polypeptide and then immobilized on a column. A solution containing candidate compounds is then passed through the column, and compounds that bind the mutated microbial polypeptide are identified. To isolate the compound, the column is washed to remove non-specifically bound molecules, and the compounds of interest are released from the column and collected. Compounds that bind the first mutated microbial polypeptide may optionally be assayed in additional columns against other mutated microbial polypeptides. Compounds that are identified as binding to one or more mutated microbial polypeptides with an affinity constant less than or equal to 10 mM are considered particularly useful in the invention.

In another method, a mutated microbial polypeptide is incubated with one or more candidate compounds and binding is detected by liquid chromatography mass spectrometry (LCMS), nuclear magnetic resonance spectroscopy (NMR) analysis, or surface plasmon resonance (i.e. Biacore technology). Binding may be determined using a radiolabeled candidate compound followed by ultrafiltration, ultracentrifugation of the mutated polypeptide-candidate compound complex, gel electrophoresis of the mutated polypeptide-candidate compound complex, equilibrium dialysis, or capillary electrophoresis. Radioactive ligand specifically bound to the receptor in preparations made from the cell line expressing the recombinant mutated microbial polypeptide can be detected in a variety of ways, including filtration of the receptor-ligand complex to separate bound ligand from unbound ligand. Alternative methods involve a scintillation proximity assay (SPA) or a FlashPlate format, in which such separation is unnecessary (see, e.g., Nakayama, Curr. Opinion Drug Disc. Dev., 1:85-91, 1998; Boss et al., J. Biomolec. Screening, 3: 285-292, 1998). Other useful assays involve the use of various enzymatic reactions including, photometric, radiometric, HPLC, and electrochemical reactions, which are described in, for example, Enzyme Assays: A Practical Approach, eds. R. Eisenthal and M. J. Danson, 1992, Oxford University Press, which is incorporated herein by reference in its entirety. Binding of fluorescent ligands can be detected in various ways, including fluorescence energy transfer (FRET), direct spectrophotofluorometric analysis of bound ligand, and fluorescence polarization (Rogers, Drug Discovery Today, 2:156-160, 1997; Hill, Cur. Opinion Drug Disc. Dev.192-97, 1998). The FRET assay, for example, may be performed by: (a) providing a mutated microbial polypeptide of the invention or a suitable polypeptide fragment thereof, either of which is coupled to a suitable FRET donor (e.g., nitro-benzoxadiazole (NBD)); (b) labeling a candidate compound with a FRET acceptor (e.g., rhodamine); (c) contacting the acceptor-labeled candidate compound and the donor-labeled mutated microbial polypeptide; and (d) measuring fluorescence resonance energy transfer. Quenching and FRET assays are related. Either one of these assays may be applied in a given case, depending on which pair of fluorophores is used in the assay.

A further method for identifying compounds that bind mutated microbial polypeptides is described in Wieboldt et al. (Anal. Chem., 69:1683-1691, 1997), incorporated herein by reference in its entirety. This technique screens combinatorial libraries of 20-30 agents at a time in solution phase for binding to a target polypeptide. Candidate compounds that bind the target polypeptide are separated from other library components by simple membrane washing. The specifically selected molecules that are retained on the filter are subsequently liberated from the target polypeptide and analyzed by high-pressure liquid chromatography (HPLC) and pneumatically-assisted electrospray (ion spray) ionization mass spectroscopy. This procedure selects library components with the greatest affinity for the target polypeptide, and may be particularly useful for small molecule libraries.

Binding may also be detected using competitive screening assays in which proteins (e.g., neutralizing antibodies) capable of binding a mutated microbial polypeptide of the invention specifically compete with a candidate compound for binding to the polypeptide. For example, a candidate compound may be contacted with two polypeptides, the first polypeptide being a mutated microbial polypeptide of the invention (e.g., any one of the mutants described herein) and the second polypeptide being a polypeptide that binds the first polypeptide under conditions that allow binding. In this respect, the second polypeptide may be any polypeptide that under normal conditions binds the first polypeptide, or alternatively, may be an antibody or an antibody fragment. For example, a candidate compound may be contacted in vitro with RpoB containing an H481D mutation and an antibody specific to this protein. Under the appropriate conditions, the mutated RpoB binds the antibody. According to this particular screening method, the interaction between these two proteins is measured following the addition of a candidate compound. A decrease in the binding of the first polypeptide to the second polypeptide following the addition of the candidate compound (relative to such binding in the absence of the compound) would identify the candidate compound as having the ability to bind the first protein and as having antimicrobial properties. Contacting of the candidate compound with the two proteins may occur in a cell-free system or using a yeast two-hybrid or three-hybrid system. If desired, the first polypeptide or the candidate compound may be immobilized on a support as described above or may have a detectable group. Alternatively, the candidate compound may be expressed on the surface of a phage or may be expressed using RNA display according to standard methods. Radiolabeled competitive binding studies are described in, for example, Lin et al. (Antimicrob. Agents Chemother., 41:2127-2131, 1997), the disclosure of which is incorporated herein by reference in its entirety. Optionally, binding may also be determined using competitive binding assays by displacing radiolabeled antibiotic, for example, by displacing rifampin or rifalazil with another unlabeled ansamycin.

Binding between a candidate compound and a mutated microbial polypeptide may also be determined by measuring the intrinsic fluorescence of the mutated microbial polypeptide and determining whether the intrinsic fluorescence is modulated in the presence of a candidate compound. Accordingly, fluorescence of the mutated microbial polypeptide is measured and compared to the fluorescence intensity of the mutated microbial polypeptide in the presence of candidate test compound, such that a decrease in fluorescence intensity indicates binding of the test compound to a mutated microbial polypeptide. Exemplary techniques are described in “Principles of Fluorescence Spectroscopy” by Joseph R. Lakowicz, New York, Plenum Press, and “Spectrophotometry And Spectrofluorometry” by C. L. Bashford and D. A. Harris Oxford, Washington, D.C., IRL Press, 1987, each of which is incorporated herein by reference in its entirety.

Another screening method to identify direct binding of compounds to a mutated microbial polypeptide relies on the principle that proteins generally exist as a mixture of folded and unfolded states, and continually alternate between the two states. When a candidate compound binds to the folded form of a mutated microbial polypeptide, the target protein molecule bound by the ligand remains in its folded state. Thus, the folded mutated microbial polypeptide is present to a greater extent in the presence of a compound that binds the mutated microbial polypeptide than in the absence of an interacting compound. Binding of the compound to the mutated microbial polypeptide can be determined by any method that distinguishes between the folded and unfolded states of the mutated microbial polypeptide (e.g., as described by Canet et al., Biophysical Journal, 80:1996-2003, 2001).

In another example, candidate compounds previously arrayed in the wells of a multi-well plate are incubated with the labeled mutated microbial polypeptide. Following washing, the wells with bound, labeled polypeptide are identified. Data obtained using different concentrations of mutated microbial polypeptides are used to calculate values for the number, affinity, and association of the polypeptide with the candidate compounds. If desired, the candidate compounds may be labeled instead of the mutated microbial polypeptide. Similarly, the mutated microbial polypeptide may be immobilized, e.g., in wells of a multi-well plate or on a solid support, and soluble compounds are then contacted with the mutated microbial polypeptide. Upon removal of unbound compound, the identity of bound candidate compounds is ascertained. Alternatively, interaction of unlabeled mutated microbial polypeptides may be detected using direct or indirect antibody labeling. Compounds that bind are considered to be candidate modulators of mutated microbial polypeptide biological activity.

Assays Measuring Biological Activity

Candidate compounds that interact with a mutated microbial polypeptide may also be identified based on their ability to reduce or inhibit the biological activity of the mutated microbial polypeptides of the invention in in vitro or in vivo assays (e.g., including animal models). Candidate compounds are contacted with a mutated microbial polypeptide having some level of a characteristic biological activity; the exact level of activity is unimportant and may be at least 10%, 20%, 30%, 40%, 50%, 60%, 70%, 80%, 90%, 100%, or more than 100% of the biological activity of the naturally-occurring, wild-type microbial polypeptide. Candidate compounds that reduce the biological activity of a mutated microbial polypeptide by at least 10%, 20%, 30%, 40%, 50%, 60%, 70%, 80%, 90%, 95%, or even 100% relative to an untreated control not contacted with the candidate compound are identified as compounds having antimicrobial activity against drug resistant microbial pathogens. Desirably, the candidate compound is contacted with a plurality of such polypeptides. This compound is identified as having antimicrobial activity against drug resistant microbial pathogens if it inhibits the biological activity of at least 1, 2, 3, 4, 5, 10, or more than 10 mutated microbial polypeptides. The identified compound may, but need not, also reduce the biological activity of the wild-type polypeptide by at least 10%, 20%, 30%, 40%, 50%, 60%, 70%, 70%, 80%, 95%, or even 100% relative to an untreated control.

In one example, a cell (e.g., a bacterial or fungal cell) expressing the mutated microbial polypeptide (e.g., any of the RpoB mutated polypeptides described herein) may be contacted with a candidate compound, after which the biological activity (e.g., RNA polymerase activity) of the microbial polypeptide is measured in the cell. In another example, contacting between candidate compounds and mutated microbial polypeptides occurs in a cell-free system or in an animal, and biological activity is then determined. Biological activity may be determined using any standard method, including those described herein. A candidate compound that reduces such biological activity relative to that of the same polypeptide in a cell not contacted with the candidate compound, identifies the candidate compound as an antimicrobial polypeptide.

To assess a change of biological activity for a mutated RpoB, for example, the IC₅₀ value may be determined using RNA polymerase assays. In these assays, cells are first permeabilized, contacted with the candidate compound, and exposed to radiolabelled RNA polymerase substrates, after which the biological activity of RNA polymerase is determined using any method known in the art or described herein. As a specific example, bacterial cells expressing mutated RpoB are first permeabilized by treatment with crushed ice or with toluene (Fisher et al., 1975. Ribonucleic acid synthesis in permeabilized mutant and wild type cells of Bacillus subtilis. In “Spores VI” (P. Gerhardt, R. N. Costilow and H. L. Sadoff, eds., American Society for Microbiology, Washington, D.C.). pp. 226-230). The candidate compound is next added to the cell culture media along with radiolabelled substrates and RpoB activity is measured.

A number of assays that employ molecular beacon probes may also be used to measure the biological activity of RNA polymerase. Molecular beacon probes are single-stranded oligonucleotide hybridization probes that form a stem-and-loop structure and that have a reporter dye attached on one end and a quencher attached at the other end. These probes typically range between 10 and 30 nucleotides, preferably between 15 and 25 nucleotides, and more preferably between 17 and 23 nucleotides. In contrast to linear oligonucleotide probes, molecular beacons contain a target-binding domain, flanked by two complementary short arm sequences. The length of these arms ranges between 4-10 nucleotides and preferably between 5-7 nucleotides. Because these arms are complementary to each other, the molecular beacon sequence forms a hairpin-loop structure. The sequence of the flanking complimentary arms may be independent of the target-binding domain sequence. Alternatively, the molecular beacon may be designed such that one arm participates both in stem formation (i.e. when the beacon is closed) and in target hybridization (i.e. when the beacon is open) (Tsourkas et al., Nucleic Acids Res. 30:4208-4215, 2002). Exemplary fluorophores include 5-Carboxyfluorescein (FAM), 6-hexachlorofluorescein (HEX), 6-Tetrachlorofluorescein (TET), Cy2, Cy3, Cy3.5, Cy5, Cy5.5, and Texas red-X. Typically, the quencher that is employed is dependent on the emission spectra of the fluorophore (see Marras et. al., Nucleic Acids Res. 30:e122, 2002). For example, FAM is typically covalently attached to the 5′ end of the oligonucleotide with Dabcyl as the preferred quencher at the 3′ end.

In solution or in the absence of a target-domain sequence, the close physical proximity of the fluorophore and quencher allows energy transfer from the donor (e.g. FAM) to the quencher (e.g. Dabcyl). Since the absorption spectra of the quencher is selected to overlap with the emission spectra of the fluorophore, the emitted electrons are captured and there is little or no fluorescence detected. When a probe hybridizes to a complementary nucleic acid strand containing a target sequence, however, the stem loop configuration is disrupted and the fluorophore and quencher are separated allowing the escape of the emitted electrons and emission of fluorescence. The rigidity and length of the probe-target hybrid precludes the simultaneous stable existence of the stem hybrid. Molecular beacon probes are designed so that their sequence is long enough for a perfectly complementary probe-target hybrid to be more stable than the stem loop configuration. The molecular beacon probes therefore spontaneously form fluorescent probe-target hybrids.

FIG. 2 is a schematic diagram illustrating one assay that utilizes such probes to detect RNA polymerase biological activity by measuring the production of RNA transcripts (as described by Liu et al. supra). In this assay, the molecular beacon probe is designed such that its “arms” share complementarity to the RNA transcript to be detected. This probe is added to a test solution in which transcription is to be detected. If RNA transcripts to which the molecular beacon probes are complementary are produced, the transcripts bind the probes and fluorescence is emitted. If no transcription is occurring, the probes remain in their stem-loop conformation.

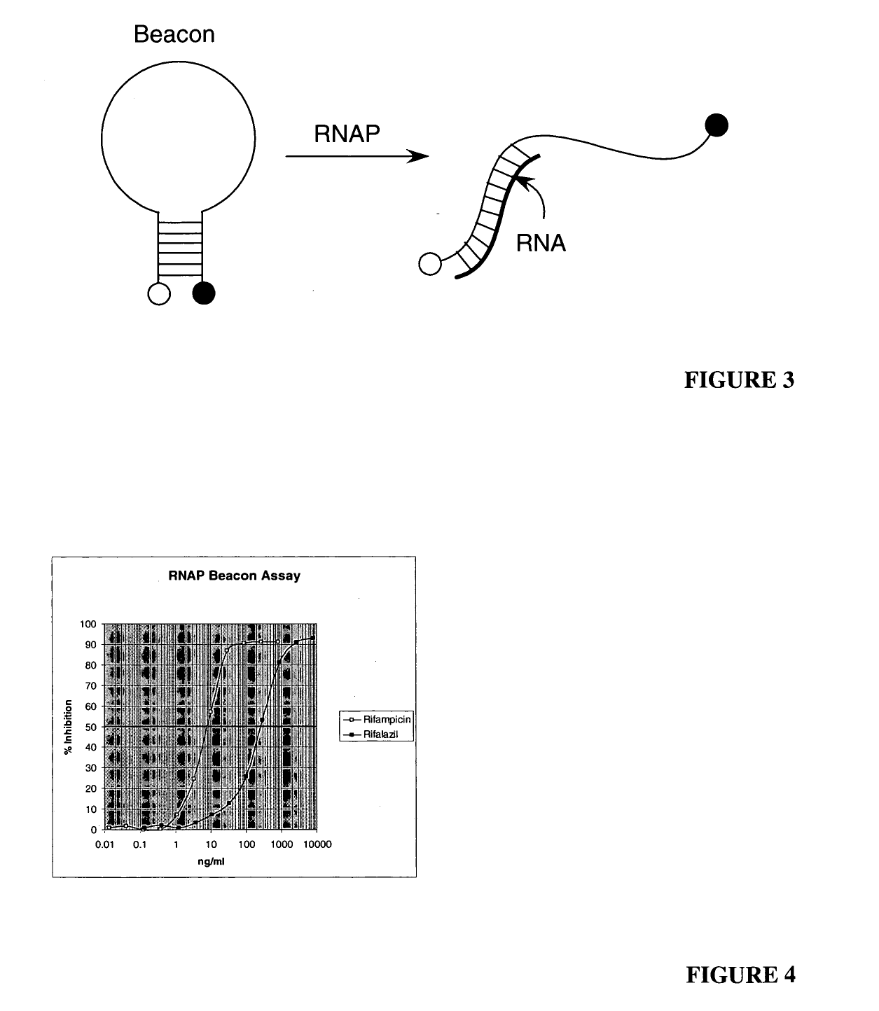

Here, we have developed a new method to detect RNA polymerase activity using the molecular beacon probes described above. The principle of this assay is depicted in FIG. 3. One important feature of this method is that the molecular beacon probe functions as both the target nucleotide sequence and the detecting species. Thus, in contrast to the assay described above (illustrated in FIG. 2), this assay does not rely on an additional molecule for a DNA template, from which RNA transcripts are to be produced, since the molecular beacon probe itself functions as such. In the presence of a biologically active RNA polymerase, a short complementary RNA transcript is produced using the probe as a template. Because the transcript shares complementarity to one of the arms of the probe, it hybridizes to that arms thereby causing the beacon to unfold and emit fluorescence. A biologically inactive RNA polymerase polypeptide, however, would not produce any RNA transcript from the probe. As a result, the probe would remain unfolded and would not emit any fluorescence. FIG. 4 shows that a bacterial RNA polymerase may be specifically inhibited and detected using the present approach. Accordingly, our method is useful for the identification of candidate compounds that inhibit the growth of drug resistant microbial species.

Alternatively, RNA polymerase-dependent in vivo transcription may be determined by measuring the incorporation of radiolabeled uracil and comparing the level of inhibition of transcription to inhibition levels for other macromolecule synthetic processes, such as DNA synthesis, protein synthesis, or cell wall synthesis (Singh et al., Antimicrob. Agents Chemother. 44:2154-9, 2000).

Assays Measuring Cell Growth

Candidate compounds of the present invention may also be identified based on their ability to reduce or inhibit the growth of microbial pathogens that express one of a panel of target mutant microbial polypeptides. For example, a candidate compound may be contacted with a plurality of cell populations, such that each contacting event is segregated from the others. Each population of cells expresses a mutated microbial polypeptide. A candidate compound that reduces or inhibits the growth of at least two populations of cells expressing mutated polypeptides, relative to the growth of control populations not contacted with the candidate compound, is identified as a compound having antimicrobial activity against drug resistant pathogens. Compounds may be screened by measuring their minimum inhibitory concentration (MIC), using standard MIC in vitro assays (see, for example, Suchland et al., Antimicrob. Agents Chemother. 47:636-642,2003; Tomioka et al., Antimicrob. Agents Chemother. 37:67, 1993; Lee et al., Am. Rev. Respir. Dis. 136:349, 1987).

Optionally, assays measuring cell growth may also be employed to confirm that an antimicrobial compound identified by any of the other assays of the invention can effectively reduce the growth of resistant microbial organisms that express the mutated microbial polypeptides.

If the contacting event occurs in vivo, the antimicrobial activity of the candidate compound may be assessed by determining the survival of treated animals relative to untreated animals, the microbial load in treated animals relative to untreated animals, or both.

The following examples are meant to illustrate the invention. They are not meant to limit the invention in any way.

### EXAMPLE 1

### Identification of Drug-Resistant RpoB Mutants

S. aureus is one of the most frequently encountered Gram-positive pathogens. Drug resistance-conferring mutations typically occur within the β subunit of RNA polymerase (RpoB mutations), in the rifampin-binding site. We have identified a collection of rifampin-resistant S. aureus mutants, as shown in Table 2. Mutants of S. aureus were isolated by inoculating a culture of S. aureus ATCC strain 29213 (standard susceptibility testing strain) into medium containing rifampin or rifalazil; by inoculating S. aureus ATCC strain 29213 into medium containing chemical derivatives of rifamycin (NCEs); by inoculating S. aureus Smith, a variant optimally adapted to colonizing and causing disease in the mouse septicemia model, into medium containing rifampin; and by using the Ian Chopra collection, the parent strain of which is S. aureus 8325-4 and described previously by Oliva et al. (Antimicrob. Agents Chemother. 45:532-9, 2001). Mutants resistant to rifampin, rifalazil, or NCEs were selected either on drug-containing plates or in liquid culture.

The mutations identified in S. aureus were all located in analogous positions in other different microbial species (as described by Wichelhaus et al., Antimicrob. Agents Chemother. 43:2813-6, 1999; Wichelhaus et al., J. Antimicrob. Chemother. 47:153-6, 2001; Yang et al., J. Antimicrob. Chemother. 42:621-8, 1998; Park et al., Int. J. Tuberc. Lung Dis. 6:166-70, 2002; Moghazeh et al., Antimicrob. Agents Chemother. 40:2655-7, 1996; Williams et al., Antimicrob. Agents Chemother. 42:1853-7, 1998; Heep et al., Eur. J. Clin. Microbiol. Infect. Dis. 21:143-5, 2002, Oliva et al., supra). We have therefore generated similar panels with other microbial species, such as Escherichia coli, Bacillus subtilis, and Chlamydia trachomatis.

The restricted number of mutations resulting in amino acid changes in RpoB (as revealed by DNA sequencing) confirmed the comprehensive nature of the collection of mutants found in Table 2. Furthermore, an exhaustive search for additional mutated positions was not successful (Table 2). The introduction of such mutations into the RpoB gene in E. coli (Garibyan et al., NA Repair (Amst). 2:593-608, 2003) and in B. subtilis (Boor et al. J. Biol. Chem. 270:20329-36, 1995) was sufficient to confer strong resistance to rifampin. Accordingly, we concluded that the mutations in RpoB were responsible for the drug resistance phenotype.

TABLE 2
S. aureus RpoB mutants

| | Strain | number | | |
|---|---|---|---|---|
| Mutation* | background(s)** | isolated | MIC Rif | MIC Rfz |
| H481Y | 1, 2, 3 | 99 | >8 | >8 |
| Q468K | 1 | 31 | >8 | >8 |
| S464P | 1, 2, 4 | 9 | >8 | 1 |
| A477D | 1, 4 | 6 | >8 | 2-4 |
| Q468R | 1 | 4 | >8 | >8 |
| H481D | 1, 4 | 4 | >8 | >8 |
| S486L | 1, 2, 4 | 26 | >8 | >8 |
| I527P | 1 | 1 | 4 | 1 |
| R484H | 1, 3 | 15 | >8 | 8 |
| R484S | 1 | 1 | 8 | 0.5 |
| R484C | 3 | 3 | 2 | 0.5 |
| H481N | 1, 4 | 2 | 2 | 0.125 |
| I527M | 1 | 1 | 0.25 | 0.063 |
| A477V | 1, 2 | 10 | 1 | 0.063 |
| Q137L | 1, 4 | 2 | 0.25 | <0.031 |
| D471V | 3 | 1 | >8 | 2 |
| D471Y | 1, 2, 4 | 5 | 1 | 0.25 |
| D471G | 1, 2 | 8 | >8 | >8 |
| H481R | 3 | 1 | >8 | >8 |
| L466S | 1, 2, 4 | 3 | 0.25 | 0.016 |
| D471E | 4 | 1 | 0.25 | <0.031 |
| None** | | — | 0.015 | 0.015 |
| total | | 233 | | |

**Wild-type parent strains for these mutants include (1) ATCC S. aureus 29213, (2) S. aureus Smith, (3) S. aureus W59536 (G. Drusano laboratory), and (4) S. aureus 8325-4 (I. Chopra laboratory). All have the same MIC values for rifampin and rifalazil.

TABLE 3
MICs of NCEs against several S. aureus strains resistant to rifampin.

| | Mutant | SA-003 | SA-004 | SA-042 | SA-044 | SA-045 | SA-047 | SA-049 | |
|---|---|---|---|---|---|---|---|---|---|
| Compound | Score | H481Y^{a} | Q468K | S486L | D471Y | S464P | A477D | H481D | Deacetylated |
| 1 | 2 | 4 | 4 | 2 | ≦0.031 | ≦0.031 | 0.25 | 2 | |
| 2 | 0 | 2 | 4 | 2 | 0.125 | 2 | 0.5 | 2 | yes |
| 3 | 0 | 2 | 4 | 2 | 0.5 | 4 | 0.5 | 4 | yes |
| 4 | 2 | 4 | 4 | 2 | 0.063 | 0.125 | 0.5 | 2 | |
| 5 | 2 | 2 | 2 | 2 | 0.5 | 0.5 | 0.25 | 1 | yes |
| 6 | 0 | 4 | 4 | 2 | ≦0.031 | ≦0.031 | 0.25 | 2 | |
| 7 | 2 | 2 | 4 | 1 | ≦0.031 | ≦0.031 | 0.125 | 1 | |
| 8 | 0 | 2 | 4 | 4 | 0.125 | 2 | 0.5 | 2 | yes |
| 9 | 0 | 4 | 4 | 2 | 0.063 | ≦0.031 | 0.125 | 2 | |
| 10 | 0 | 4 | 4 | 2 | ≦0.031 | 1 | 0.25 | 2 | yes |
| 11 | 2 | 2 | 4 | 2 | 0.063 | 0.063 | 0.25 | 2 | |
| 12 | 2 | 4 | 4 | 2 | ≦0.031 | 0.063 | 0.25 | 2 | |
| 13 | 0 | 4 | 4 | 2 | 0.5 | 1 | 0.5 | 2 | yes |
| 14 | 2 | 2 | 4 | 2 | ≦0.031 | 0.063 | 0.5 | 4 | |
| 15 | 1 | 2 | 4 | 1 | 0.5 | 1 | 0.5 | 2 | yes |
| 16 | 2 | 2 | 2 | 4 | ≦0.031 | 0.063 | 0.25 | 2 | |
| 17 | 2 | 1 | 2 | 1 | 0.125 | 0.5 | 0.125 | 1 | yes |
| 18 | 0 | 4 | 4 | 1 | 0.063 | 0.063 | 0.25 | 4 | |
| 19 | 1 | 2 | 4 | 2 | ≦0.031 | 0.063 | 0.25 | 4 | |
| 20 | 1 | 2 | 4 | 2 | 0.125 | 0.5 | 0.125 | 2 | yes |
| 21 | 2 | 2 | 4 | 2 | ≦0.031 | ≦0.031 | 0.25 | 2 | |

^{a}RpoB mutation

Having identified these mutants, the minimum inhibitory concentrations (MIC) of a number of rifamycin derivatives were determined in microtiter trays by inoculating 1-8×10⁴ microorganisms in 100 μl of Mueller-Hinton Broth (cation adjusted) containing the indicated compound (Table 3) (National Committee for Clinical Laboratory Standards, Methods for Dilution Antimicrobial Susceptibility Tests for Bacteria That Grow Aerobically-Fourth Edition: Approved Standard M7-A4. NCCLS, Villanova, Pa., 1997). These cultures were incubated for 16-20 hours at 35° C. Based on our results, the most important mutants (conferring the strongest resistance) were identified for more extensive testing of NCEs. These mutants were H481Y, Q468K, S486L, D471Y, S464P, A477D, and H481D. The MICs of a number of candidates, all of which had an MIC at or below 0.015 μg/ml (the MIC of rifalazil) were determined in the same manner. The MICs showed a consistent pattern; compounds that showed superior MICs against mutant H481Y also showed MICs that were improved for the entire panel of mutants. In general, the panel of mutants H481Y, Q468K, S486L, D471Y, S464P, A477D, and H481D, which showed the highest MICs against all compounds, are mutant RpoB polypeptides that may be utilized in the screening methods of the invention. An exception to the uniformity of the measure of resistance is provided by the mutants D471Y and S464P, which together have lower MICs for some of the compounds compared to the rest. Compounds that tend to have lower MICs to mutants D471Y and S464P are often compounds that contain the acetyl group at position 25 of the rifalazil molecule, rather than deacetylated compounds at position 25, in compounds that otherwise have an identical structure. Deacetylated compounds are denoted in Table 3. However, these mutants do not have the strongest effect (i.e., they do not show the highest MICs) among the members of the mutant RpoB panel. Therefore, based on the knowledge garnered from this comprehensive set of mutants, it is possible to define a sub-population of mutants that are the most important to evaluate extensively for interactions between mutant RNA polymerase and rifamycin derivatives.

A further confirmation of the validity of MIC testing of the mutant panel was provided by our mutant score test. Using this strategy, compounds were individually tested for resistance development by inoculating 10⁹ cells of S. aureus 29213, a standard susceptibility strain, onto a large agar plate (150 mm) containing Mueller Hinton Agar as well as 1 μg/ml of the test compound. The presence of rifalazil or rifampin in these plates generally allowed for the growth of ˜50 colonies, all colonies representing a mutant sub-population or populations in the culture. Some compounds prevented the growth of any mutant colonies (mutant score of 2), while the presence of other compounds allowed the growth of <10 colonies (mutant score of 1). Most compounds failed to prevent the growth of mutant colonies on the plate (a mutant score of 0). The MICs of NCEs on agar plates were lower than MICs determined by growth in liquid broth. However, compounds that score 2 in this test consistently showed favorable low MICs against the mutant panel. This correlation provided additional confidence in the value and ranking of compounds by MIC testing of the mutant panel, as compounds having a score of 2 have tracked with strong activity in MIC testing against resistant strains, as indicated in Table 3.

### EXAMPLE 2

### In vitro Screening Using Mutant RpoB Polypeptides

Antimicrobial compounds may be identified by screening for interactions with mutated RpoB polypeptides in vitro. In one particular assay, various mutated RpoB polypeptides from S. aureus are immobilized in the wells of a multi-well plate such that each different mutant is present in its own well. A plurality of labeled candidate compounds are then individually contacted with each mutated RpoB polypeptide such that each candidate compound-polypeptide contacting event is segregated from the others. After a sufficient time to allow for binding, unbound compound is removed by washing and the presence of bound compound is determined by detection of the label. Candidate compounds that bind mutant RpoB polypeptides are thus identified.

In this example, compounds identified as binding RpoB are next optimally tested for their ability to reduce RNA polymerase activity. Each of the compounds identified as binding one or more RpoB polypeptides is incubated in a solution containing the folded beacon shown in FIG. 3 in the presence of various active bacterial RNA polymerases, each having a mutated RpoB polypeptide. As above, each RNA polymerase candidate compound is contacted with the polypeptide separately and distinctly from the other RNA polymerase candidate compounds. Functional RNA polymerase polypeptides bind the folded beacon and transcribe a short complementary RNA fragment, thereby causing the beacon to unfold and emit a fluorescent signal. Samples in which a fluorescent signal is emitted are considered to contain a functional RNA polymerase. Samples having a reduced signal (compared to an untreated control) are considered to contain a candidate compound having the ability to inhibit RNA polymerase activity. Candidate compounds that inhibit the biological activity of at least two mutated RNA polymerases are considered to be particularly desirable.

### EXAMPLE 3

### In vivo Screening of Rifamycin Derivatives

Antimicrobial compounds may also be screened in vivo using a mouse septicemia model. In one particular example, mice were inoculated with an S. aureus Smith strain (Weiss) encoding a mutated microbial polypeptide (e.g., a mutated RpoB). Compounds were administered either IV or orally 30 minutes following inoculation, and observations was continued for three days. Compounds that promoted the survival of inoculated mice were identified as being compounds that are effective against antibiotic-resistant forms of S. aureus.

In one example, when mutant strains derived from S. aureus Smith containing the RpoB H481Y alteration were inoculated in the mouse model by IV or oral route, two compounds, compound 15 and compound 16 from Table 3, were found to protect mice from lethality, relative to untreated animals (Table 4). The dose that was essential for efficacy was found to be considerably higher for the mutant strains than for the wild-type S. aureus Smith. This was accounted for by the increased MIC against these mutant strains compared with the wild-type strain (MICs of 2 μg/ml and 0.004-0.008 μg/ml, respectively). Similar results were observed for mutants containing the S486L and L466S mutations in RpoB, which also conferred strong resistance when tested by IV administration (Table 4).

According to our results, candidate compounds that showed significant MICs against mutant cells in culture also proved efficacious in vivo against mutant strains, whereas rifampin failed to be effective against mutant strains in vivo. These results demonstrate the efficacy of the screening assays of the present invention.

TABLE 4
In vivo efficacy in the mouse septicemia model utilizing S. aureus Smith strain
carrying the indicated mutation conferring strong resistance to rifampin

| | | No Drug | 1.1 × 10⁷ | 1.4 × 10⁷ | 1.1 × 10⁷ |
|---|---|---|---|---|---|
| | | Survival | CFU/mouse | CFU/mouse | CFU/mouse |
| | | (n = 10) | 0 | 0 | 0 |
| | | Dose | Survival | Survival | Survival |
| Compound | Delivery | (mg/kg) | (n = 5) | (n = 5) | (n = 5) |
| Rifampicin | IV | 20.0 | 0 | 1 | 0 |
| | | 6.0 | 0 | 0 | 0 |
| Ciprofloxacin | IV | 6.0 | 4 | 5 | 5 |
| | | 2.0 | 5 | 3 | 4 |
| | | 0.6 | 2 | 1 | 1 |
| Compound 15 | IV | 20.0 | 1 | 5 | 3 |
| | | 6.0 | 1 | 1 | 1 |
| Compound 16 | IV | 20.0 | 4 | 3 | 5 |
| | | 6.0 | 0 | 1 | 1 |
| Rifampicin | oral | 200.0 | 0 | nd | nd |
| Compound 15 | oral | 200.0 | 5 | nd | nd |

### OTHER EMBODIMENTS

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of identifying a compound that inhibits the growth of drug resistant microbial pathogens, said method comprising the steps of:
(a) producing a derivative compound of an antimicrobial compound;
(b) contacting said derivative compound with a plurality of mutated microbial polypeptides conferring drug resistance under conditions that ensure that each contacting event is segregated from the others; and
(c) determining whether said derivative compound interacts with said mutated microbial polypeptides, wherein a derivative compound that interacts with at least two different mutated microbial polypeptides is a compound that inhibits the growth of drug resistant microbial pathogens.

2. The method of claim 1, wherein said contacting occurs in a cell-free environment.

3. The method of claim 1, wherein said contacting occurs inside a microbial cell.

4. The method of claim 1, wherein said contacting occurs in or on an animal.

5. The method of claim 1, wherein said interaction is determined by determining whether said candidate compound reduces growth of said microbial cell

6. The method of claim 1, wherein an interaction is determined by determining the biological activity of said mutated microbial polypeptides, wherein a decrease in the level of said biological activity relative to the biological activity of mutated microbial polypeptides not contacted with said derivative compound, identifies said derivative compound as being a compound that inhibits the growth of drug resistant microbial pathogens.

7. The method of claim 1, wherein an interaction is determined by determining binding of said derivative compound to a mutated microbial polypeptide.

8. The method of claim 1, wherein said mutated microbial polypeptides are mutated RpoB polypeptides.

9. The method of claim 1, wherein said mutated microbial polypeptides are fusion polypeptides.

10. A method of identifying a compound that inhibits the growth of drug resistant microbial pathogens, said method comprising the steps of:
(a) contacting at least ten candidate compounds with a plurality of mutated microbial polypeptides conferring drug resistance, under conditions that ensure that each contacting event is segregated from the others; and
(b) determining whether said candidate compounds interact with said mutated microbial polypeptides, wherein a candidate compound that interacts with at least two different mutated microbial polypeptides is identified as a compound that inhibits the growth of drug resistant microbial pathogens.

11. The method of claim 10, wherein said interaction is determined by determining whether said candidate compound reduces growth of said microbial cell.

12. The method of claim 10, wherein an interaction is determined by determining the biological activity of said mutated microbial polypeptides, wherein a decrease in the level of said biological activity relative to the biological activity of mutated microbial polypeptides not contacted with said candidate compound, identifies said candidate compound as being a compound that inhibits the growth of drug resistant microbial pathogens.

13. The method of claim 10, wherein an interaction is determined by determining binding of said candidate compound to a mutated microbial polypeptide.

14. The method of claim 10, wherein said mutated microbial polypeptides are mutated RpoB polypeptides.

15. The method of claim 10, wherein said mutated microbial polypeptides are fusion polypeptides.

16. A method of identifying a compound that inhibits the growth of drug resistant microbial pathogens, said method comprising the steps of:
(a) contacting a candidate compound with a plurality of mutated microbial polypeptides conferring drug resistance under conditions that ensure that each contacting event is segregated from the others; and
(b) determining whether said candidate compound binds said mutated microbial polypeptides, wherein a candidate compound that binds at least two different mutated microbial polypeptides is a compound that inhibits the growth of drug resistant microbial pathogens.

17. The method of claim 16, wherein said mutated microbial polypeptides are mutated RpoB polypeptides.

18. The method of claim 16, wherein said mutated microbial polypeptides are fusion polypeptides.

19. A method of identifying a compound that inhibits the growth of drug resistant microbial pathogens, said method comprising the steps of:
(a) contacting a candidate compound with a plurality of mutated microbial polypeptides conferring drug resistance under conditions that ensure that each contacting event is segregated from the others; and
(b) determining in vitro whether said candidate compound reduces the biological activity of said mutated microbial polypeptides, wherein a candidate compound that reduces the biological activity of at least two different mutated microbial polypeptides relative to the biological activity of mutated microbial polypeptides not contacted with said candidate compound is identified as a compound that inhibits the growth of drug resistant microbial pathogens.

20. The method of claim 19, wherein said mutated microbial polypeptides are mutated RpoB polypeptides.

21. The method of claim 19, wherein said mutated microbial polypeptides are fusion polypeptides.

22. A method of identifying a compound that inhibits the growth of drug resistant microbial pathogens, said method comprising the steps of:
(a) contacting a candidate compound with a mutated microbial polypeptide conferring drug resistance in vitro;
(b) determining whether said candidate compound interacts with said mutated microbial polypeptide, and continuing to step (c) if said candidate compound interacts with said mutated microbial polypeptide;
(c) contacting said candidate compound identified in step (b) with a mutated microbial polypeptide in an animal; and
(d) determining whether said candidate compound interacts with said mutated microbial polypeptide in said animal, wherein a candidate compound that interacts with said mutated microbial polypeptide in said mammal is identified as a compound that inhibits the growth of drug resistant microbial pathogens.

23. The method of claim 22, wherein said mutated microbial polypeptides are mutated RpoB polypeptides.

24. The method of claim 22, wherein said mutated microbial polypeptides are fusion polypeptides.

25. A method of identifying a compound that inhibits the growth of drug resistant microbial pathogens, said method comprising the steps of:
(a) contacting a candidate compound with a mutated microbial polypeptide conferring drug resistance;
(b) determining whether said candidate compound interacts with said mutated microbial polypeptide and continuing to step (c) if a candidate compound is identified as having the ability to interact with said mutated microbial polypeptide;
(c) contacting said candidate compound identified in step (b) with a plurality of mutated microbial polypeptides conferring drug resistance under conditions that ensure that each contacting event is segregated from the others; and
(d) determining whether said candidate compound interacts with said mutated microbial polypeptides, wherein a candidate compound that interacts with at least two different mutated microbial polypeptides is identified as a compound that inhibits the growth of drug resistant microbial pathogens.

26. The method of claim 25, wherein said mutated microbial polypeptides are mutated RpoB polypeptides.

27. The method of claim 25, wherein said mutated microbial polypeptides are fusion polypeptides.

28. A surface comprising a plurality of mutated microbial polypeptides conferring drug resistance, wherein said mutated microbial polypeptides are arranged on said surface such that, when contacted with a candidate compound, each polypeptide-candidate compound contacting event is segregated from the others.

29. The surface of claim 28, wherein said polypeptides are immobilized on said surface.

30. The surface of claim 28, wherein said mutated microbial polypeptides are mutated RpoB polypeptides.

31 A plurality of chromatographic columns, wherein each column comprises a mutated microbial polypeptide conferring drug resistance immobilized on said column.

32. The plurality of chromatographic columns of claim 31, wherein said mutated microbial polypeptides are mutated RpoB polypeptides.

33. A method of measuring RNA polymerase biological activity, said method comprising the steps of:
(a) providing an oligonucleotide that has a stem region comprising a double stranded segment formed between two complementary nucleotide sequences under suitable conditions, wherein the formation of said double stranded segment reduces or inhibits the emission of fluorescence from said fluorophore and wherein said oligonucleotide is covalently linked to a quencher and a fluorophore, wherein said quencher is linked to the 5′ or 3′end of said oligonucleotide and wherein said fluorophore is linked to the other end; and
(b) contacting said oligonucleotide with a test sample under conditions allowing transcription of said oligonucleotide, wherein an RNA transcript produced by said transcription binds to one of the two nucleotide sequences in said double stranded segment, such that the emission of fluorescence from said fluorophore is increased;
(c) detecting the emission of fluorescence from said test sample relative to a control sample, wherein an increase in fluorescence emitted from said test sample identifies said test sample as containing RNA polymerase polypeptides having biological activity.

34. A method of identifying a compound that inhibits the growth of drug resistant microbial pathogens, said method comprising the steps of:
(a) contacting a candidate compound with a mutated RpoB polypeptide conferring drug resistance and an oligonucleotide that has a stem region comprising a double stranded segment formed between two complementary nucleotide sequences under suitable conditions, wherein the formation of said double stranded segment reduces or inhibits the emission of fluorescence from said fluorophore and wherein said oligonucleotide is covalently linked to a quencher and a fluorophore, wherein said quencher is linked to the 5′ or 3′end of said oligonucleotide and wherein said fluorophore is linked to the other end and wherein said contacting occurs under conditions that allow transcription of said oligonucleotide, wherein an RNA transcript produced by said transcription binds to one of the two nucleotide sequences in said double stranded segment, such that the emission of fluorescence from said fluorophore is increased
(b) detecting the emission of fluorescence from said contacting event relative to an untreated control, wherein a decrease in fluorescence emitted from said contacting event identifies said candidate compound as having the ability to reduce RNA polymerase biological activity, thereby identifying said candidate compound as being a compound that inhibits the growth of drug resistant microbial pathogens.

35. A method of identifying a compound that inhibits the growth of drug resistant microbial pathogens, said method comprising the steps of:
(a) contacting a candidate compound with a plurality of mutated RpoB polypeptides conferring drug resistance and an oligonucleotide that has a stem region comprising a double stranded segment formed between two complementary nucleotide sequences under suitable conditions, wherein the formation of said double stranded segment reduces or inhibits the emission of fluorescence from said fluorophore and wherein said oligonucleotide is covalently linked to a quencher and a fluorophore, wherein said quencher is linked to the 5′ or 3′end of said oligonucleotide and wherein said fluorophore is linked to the other end and wherein said contacting occurs under conditions that ensure that each contacting event is segregated from the others and under conditions that allow transcription of said oligonucleotide, wherein an RNA transcript produced by said transcription binds to one of the two nucleotide sequences in said double stranded segment, such that the emission of fluorescence from said fluorophore is increased;
(b) detecting the emission of fluorescence from each contacting event relative to a corresponding untreated control, wherein a decrease in fluorescence emitted from at least two of said contacting events identifies said candidate compound as having the ability to reduce RNA polymerase biological activity, thereby identifying said candidate compound as being a compound that inhibits the growth of drug resistant microbial pathogens.
